(19) United States
(12) Patent Application Publication
(10) Pub. No.: US 2026/0306368 A1
(43) Pub. Date: Oct. 8, 2026

(54) PRODUCTION METHOD OF OIL DISPERSION AND COSMETIC INCLUDING THIS OIL DISPERSION

(71) Applicant: NIKKO CHEMICALS CO., LTD., Tokyo (JP)

(72) Inventors: Noboru NAGATANI, Tokyo (JP); Shohei NAKAMURA, Tokyo (JP); Junya NAKANO, Tokyo (JP); Meng QIN, Tokyo (JP)

(21) Appl. No.: 19/480,068

(22) PCT Filed: Apr. 25, 2024
(86) PCT No.: PCT/JP2024/016256

§ 371 Date: Oct. 30, 2025

(30) Foreign Application Priority Data
Jun. 2, 2023 (JP) 2023-091279

(51) Int. Cl. A61K 8/04

(52) U.S. Cl. CPC A61K 8/04; A61K 2800/43

(57) ABSTRACT

A production method of an oil dispersion includes: a step (1) of adding a component (A) gradually to a mixture in which a component (C) is dissolved or dispersed in a component (B) while heating the mixture oil at a temperature of 40° C. to 120° C., and a step (2) of further mixing a dispersion to which a total amount of the component (A) is added at a temperature of 40° C. to 120° C. The component (A) is inorganic oxide powder that is not surface-hydrophobized, the component (B) is silicone oil, and the component (C) is at least one compound selected from silicone compounds, organic silicon compounds, organic titanate compounds, and higher fatty acids.

### TECHNICAL FIELD

The present invention relates to a production method of an oil dispersion and a cosmetic including the oil dispersion. More specifically, the present invention relates to a production method of an oil dispersion that can be dispersed in an oil despite its high solid content by adding inorganic oxide powder to silicone oil containing at least one compound selected from silicone compounds, organic silicon compounds, organic titanate compounds, and higher fatty acids at a specific temperature, and to a cosmetic including the oil dispersion produced by this production method.

### BACKGROUND ART

In general, oil dispersions for cosmetics are obtained by previously highly dispersing, in an oil medium, a pigment to be blended in makeup cosmetics such as foundations, eye shadows, and blushes, or basic cosmetics such as sunscreen cosmetics, hair cosmetics, milky lotions, and creams. Therefore, the oil dispersions for cosmetics are widely used because of good handling properties, such as less scattering and contamination of pigments during the preparation of cosmetics and unnecessity of special mixing equipment because the pigments are already highly dispersed.

In addition, inorganic oxide powders have a strong aggregation force between the powders, which makes them difficult to be dispersed in oils as is. Therefore, inorganic oxide powders used in oil dispersions are widely subjected to a hydrophobic treatment in order to facilitate dispersion of the inorganic oxide powders in oils. For example, a method, in which the pigment surface is coated with silicones such as higher fatty acids, higher alcohols, hydrocarbons, silicone oils, and silicone resins to impart hydrophobicity, is known. Among these, many methods of hydrophobic treatments of the pigments using silicones as surface treatment agents have been established up to now (see, for example, Patent Literatures 1 and 2), and oil dispersions using these methods are also widely known (see, for example, Patent Literatures 3, 4, and 5).

Furthermore, since inorganic oxide powders have a strong aggregation force between powder particles, in order to suppress reaggregation after dispersion and to maintain dispersion stability, the inorganic oxide powders are generally dispersed by adding a silicone-based dispersing agent such as polyether-modified silicone or a surfactant (see, for example, Patent Literatures 6 and 7).

### CITATION LIST

### Patent Literatures

Patent Literature 1: JP 60-163973 A
Patent Literature 2: JP 62-177070 A
Patent Literature 3: JP 2002-80748 A
Patent Literature 4: JP 2005-171145 A
Patent Literature 5: JP WO 2011/007668 A1
Patent Literature 6: JP 2007-291379 A
Patent Literature 7: JP 2012-184178 A

### SUMMARY OF INVENTION

However, in the method of hydrophobically treating inorganic oxide powder, an oil dispersion can be prepared only after the inorganic oxide powder is first subjected to a step of surface-treatment with a surface treatment agent, which is a problem in terms of complexity.

In addition, when an oil dispersion obtained using a dispersing agent, a surfactant, or the like is included in a cosmetic, it may have adverse effects such as deteriorating the storage stability or makeup durability of the cosmetic.

The present invention has been made in consideration of the above-mentioned problems, and aims to provide a production method that can obtain an oil dispersion having excellent dispersibility of inorganic oxide powder without requiring a previous surface treatment to enhance the dispersibility of the inorganic oxide powder in an oil and without the addition of a dispersing agent, and a cosmetic including the oil dispersion produced by this production method.

As a result of intensive research conducted by the present inventors to achieve the above object, they found that an oil dispersion having a high dispersibility of pigment is obtained and that a high dispersibility of pigment can be maintained even when no dispersing agent is used by adding inorganic oxide powder that is not surface-hydrophobized to silicone oil including at least one compound selected from the group consisting of silicone compounds, organic silicon compounds, organic titanate compounds, and higher fatty acids as a treatment agent under heating at a temperature of 40° C. to 120° C., and they completed the present invention.

The production method of an oil dispersion according to one embodiment of the present invention is characterized by including: a step (1) of adding inorganic oxide powder while silicone oil including at least one compound selected from silicone compounds, organic silicon compounds, organic titanate compounds, and higher fatty acids is heated at a temperature of 40° C. to 120° C.; and a step (2) of further mixing a dispersion to which the total amount of the inorganic oxide powder has been added, at a temperature of 40° C. to 120° C.

In the production method of the oil dispersion of the present invention, a kneading mixer or a wet mixing disperser is preferably used for dispersing in the step (2) of the above production method.

One aspect of the production method of the oil dispersion of the present invention is a method for producing an oil dispersion containing the following components (A) to (C), and is a production method of an oil dispersion, which includes: a step (1) of adding a component (A) to a mixture in which a component (C) is dissolved or dispersed in a component (B) while heating the mixture at a temperature of 40° C. to 120° C., and a step (2) of further mixing a dispersion to which a total amount of the component (A) is added at a temperature of 40° C. to 120° C.; wherein, after all steps of the production method, the oil dispersion is cooled, and is further dispersed using a wet mixing disperser:
(A) inorganic oxide powder that is not surface-hydrophobized,
(B) silicone oil, and
(C) at least one compound selected from the group consisting of silicone compounds, organic silicon compounds, organic titanate compounds, and higher fatty acids.

In the production method of the oil dispersion of the present invention, the production is preferably performed by cooling the oil dispersion preferably to room temperature (20° C. to 30° C.) and further additionally dispersing it using a wet mixing disperser, after the step (2) of the above production method.

Moreover, the cosmetic of the present invention is a cosmetic characterized by including the oil dispersion produced by the production method of the present invention.

### DESCRIPTION OF EMBODIMENTS

Next, the specific embodiments of the production method of an oil dispersion according to the present invention and the cosmetic containing the oil dispersion produced by this production method will be described. In this specification, “X to Y” indicating a range means “X or more, Y or less”. Furthermore, unless otherwise specified, operations are performed under the conditions of room temperature (20° C. to 30° C.)/relative humidity of 45 to 55% RH. Furthermore, it should be understood that all combinations of the embodiments are disclosed in the present application.

The production method of the oil dispersion of the present invention is a method for producing an oil dispersion containing the following components (A) to (C):
(A) inorganic oxide powder that is not surface-hydrophobized,
(B) silicone oil, and
(C) at least one compound selected from the group consisting of silicone compounds, organic silicon compounds, organic titanate compounds, and higher fatty acids,
   and is a production method of an oil dispersion, which includes: the step (1) of adding the component (A) to a mixture in which the component (C) is dissolved or dispersed in the component (B) while heating the mixture at a temperature of 40° C. to 120° C.; and a step (2) of further mixing a dispersion to which a total amount of the component (A) is added at a temperature of 40° C. to 120° C.

According to the present invention, an oil dispersion, which has a high dispersibility of pigment and can maintain a high dispersibility after time has passed (even without using a dispersing agent), can be obtained, and furthermore a cosmetic containing it can be obtained.

(Component (A): Inorganic Oxide Powder that is not Surface-Hydrophobized)

Examples of the inorganic oxide powder that is not surface-hydrophobized, which is the component (A) of the present invention, include inorganic oxide such as silicic anhydride (silicon oxide), aluminum oxide, iron oxide (red iron oxide, yellow iron oxide, black iron oxide, or the like), titanium oxide, zinc oxide, cerium oxide, and bismuth oxide, which are generally used for cosmetics. In particular, when it is used for the purpose of an effect of shielding ultraviolet rays, fine particles of inorganic oxides, such as fine particles of titanium oxide, zinc oxide, iron oxide, cerium oxide, bismuth oxide, and the like are used. These may be used as a composite of two or more kinds thereof. These may be used alone or in combination of two or more kinds thereof.

As the inorganic oxide powder in the component (A), those that are not subjected to the hydrophobic treatment are used. Here, the hydrophobic treatment refers to a treatment with a known hydrophobic surface treatment agent such as a silicone treatment or a fatty acid treatment. In addition, the inorganic oxide powder is preferably untreated powder. The untreated powder refers to powder that is not surface-treated with a surface treatment agent. In one aspect, the component (A) is at least one kind selected from the group consisting of untreated iron oxide, untreated titanium oxide, and untreated zinc oxide.

The particle diameter of the inorganic oxide powder is, for example, 0.001 to 50 μm, and may be 0.01 to 30 μm. The particle diameter is based on volume and can be measured using a laser diffraction particle diameter analyzer.

The solid content concentration of the inorganic oxide powder that is the component (A) of the present invention in the oil dispersion (content rate of the component (A) relative to the solid content in the oil dispersion) is preferably 25% by mass or more, more preferably 25 to 80% by mass, and still more preferably 30 to 80% by mass. When the solid content concentration of the inorganic oxide powder in the oil dispersion is 25% by mass or more, the dispersibility of the inorganic oxide powder after time has passed becomes good, and when it is 80% by mass or less, aggregation of the powder is easily suppressed.

### (Component (B): Silicone Oil)

The component (B) is silicone oil. Here, the silicon oil of the component (B) is used as a dispersing medium. When the silicone oil is used as a dispersing medium, the dispersibility of the inorganic oxide powder is significantly improved. Here, in the production of the oil dispersion, when a plurality of silicone oils is used, the silicone oil having the largest amount added is considered as the silicone oil of the component (B). When there is a plurality of silicone oils having the largest content, all the silicone oils are considered as the component (B). Specifically, the silicone oil of the component (B) is preferably silicone oil in an amount of more than 50% by mass relative to the total amount of the component (B) and the component (C).

In the present invention, examples of the silicone oil of the component (B) used as the dispersing medium include dimethylpolysiloxane (dimethicone), methylphenyl polysiloxane, diphenylpolysiloxane (diphenyl dimethicone), diphenylsiloxy phenyl trimethicone, alkyl-modified silicone such as caprylyl methicone, methyl trimethicone, cyclopentasiloxane methyl trimethicone, octamethylcyclotetrasiloxane, decamethylcyclopentasiloxane, and dodecamethyl cyclohexasiloxane. As the silicone oil of the component (B), at least one kind selected from the group consisting of dimethylpolysiloxane (dimethicone), diphenylsiloxy phenyl trimethicone, decamethylcyclopentasiloxane, and methylphenyl polysiloxane is preferable, and at least one kind selected from the group consisting of dimethylpolysiloxane (dimethicone), diphenylsiloxy phenyl trimethicone, and decamethylcyclopentasiloxane is preferable.

The silicone oil of the component (B) may be either volatile or non-volatile. The viscosity of the silicone oil of the component (B) at 25° C. is preferably 50 mPa·s or less (lower limit of 1 mPa·s or more), and may be 20 mPa·s or less. When the viscosity of the silicone oil is 50 mPa·s or less, the viscosity of the dispersion is appropriate.

The content of the component (B) in the oil dispersion is preferably 25% by mass or more, more preferably 25 to 80% by mass, and still more preferably 25 to 70% by mass, in terms of the effect of the present invention.

(Component (C): At Least One Compound Selected from the Group Consisting of Silicone Compounds, Organic Silicon Compounds, Organic Titanate Compounds, and Higher Fatty Acids)

The component (C) of the present invention is at least one compound selected from the group consisting of silicone compounds, organic silicon compounds, organic titanate compounds, and higher fatty acids. The component (C) is used as a treatment agent. When the inorganic powder is added while heating in such a state that the component (C) is dissolved and dispersed in the component (B), the dispersibility of the inorganic powder is improved and good dispersibility is maintained. Here, the component (B) is excluded as the component (C).

The silicone compound that is the component (C) of the present invention is not particularly limited as long as it can be used as cosmetics. Examples thereof include methyl hydrogen polysiloxane, amino-modified silicone, polyether-modified silicone, polyglycerin-modified silicone, carboxylic acid-modified silicone, alkyl-modified and polyether-modified silicone, alkyl-modified and polyglycerin-modified silicone (for example, lauryl polyglyceryl-3 polydimethylsiloxyethyl dimethicone), pyrrolidone carboxylic acid-modified silicone, triethoxysilylethyl polydimethylsiloxyethyl dimethicone, and triethoxysilylethyl polydimethylsiloxyethyl hexyl dimethicone. Note that, although the above is included in the silicone oil, as described above, the silicone oil of the component (B) refers to the silicone oil having the highest content, and is therefore distinguished from the silicone oil of the component (C)

The silicone compound that is the component (C) is preferably liquid at normal temperature (e.g., 25° C.). Note that, the liquid refers to one having fluidity at 25° C.

Examples of the organic silicon compound that is the component (C) of the present invention include silylating agents and silane coupling agents to which an organic group is introduced. Examples thereof include: alkoxysilanes such as tetramethoxy silane, methyltrimethoxysilane, dimethyldimethoxysilane, tetraethoxysilane, methyltriethoxysilane, dimethyldiethoxysilane, isobutyltrimethoxysilane, n-decyltrimethoxysilane, n-octyltrimethoxysilane, octyltriethoxysilane, octadecyltrimethoxysilane, and octadecyltriethoxysilane; and silazanes such as dimethylpolysiloxysilazane.

Examples of the organic titanate compound that is the component (C) of the present invention include alkyl titanates of a long-chain carboxylic acid-type, a pyrophosphoric acid-type, a phosphorous acid-type, and an amino acid-type. Among these, long-chain carboxylic acid-type organic titanate compounds, which have an alkyl group having 8 to 24 carbon atoms, are preferable as the organic titanate compounds. Specific examples of the alkyl titanates include isopropyl triisostearoyl titanate, isopropyl trioctanoyl titanate, isopropyl dimethacryl isostearoyl titanate, isopropyl isostearoyl diacryl titanate, and diisostearoyl ethylene titanate.

Examples of the higher fatty acid that is the component (C) of the present invention include lauric acid, myristic acid, palmitic acid, stearic acid, isostearic acid, palmitoleic acid, oleic acid, and linoleic acid. Isostearic acid, palmitoleic acid, oleic acid, linoleic acid, and the like, which are liquid at normal temperature (e.g., 25° C.), are preferable because the viscosity of the oil dispersion can be decreased.

Among these, the component (C) is preferably at least one kind selected from the group consisting of methyl hydrogen polysiloxane, carboxylic acid-modified silicone, alkyl-modified and polyglycerin-modified silicone, pyrrolidone carboxylic acid-modified silicone, amino-modified silicone, polyglycerin-modified silicone, alkoxysilane, isopropyl triisostearoyl titanate, and higher fatty acids.

The component (C) may be used alone or in combination of two or more kinds thereof.

The amount of the component (C) blended in the oil dispersion (in the case of two or more kinds of component (C), their total amount) is preferably 1 to 15% by mass, and more preferably a range of 2 to 10% by mass, relative to the component (A). When the amount of the component (C) blended is equal to or more than the above lower limit, the inorganic oxide powder is easily uniformly dispersed in the silicone oil. In the case where the amount is equal to or less than the above upper limit, when the oil dispersion is blended in a cosmetic, the feeling in use or the stability of the cosmetic easily becomes good.

The content of the component (C) in the oil dispersion is preferably 0.5 to 10% by mass, and more preferably 1 to 8% by mass, in terms of the effect of the present invention.

Use of two or more kinds of component (C) is one preferable aspect because the viscosity of the oil dispersion of the present invention can be decreased in some cases. For example, as the component (C), a combination of the silicone compound and higher fatty acid; a combination of the organic silicon compound and higher fatty acid; and a combination of the silicone compound and the organic silicon compound are preferable aspects.

### (Other Components)

The oil dispersion of the present invention does not preferably include a component other than the components (A), (B), and (C) at such a rate that is more than 20% by mass in the oil dispersion, but may include other components at a rate of 20% by mass or less relative to the oil dispersion within such a range that the performance of the oil dispersion is not impaired. The above rate may be more preferably 10% by mass or less, and may be 5% by mass or less, 3% by mass or less, 1% by mass or less, or 0% by mass (the oil dispersion consists of the components (A), (B), and (C)). Note that, preferably, a compound or the like that may impair the stability of the oil dispersion is not added.

### (Production Method)

The production method of the oil dispersion of the present invention is the production method performed in the order of the step (1) and the step (2) described below.

### Step (1);

In the step (1), while heating a mixture of at least one compound selected from the group consisting of silicone compounds, organic silicon compounds, organic titanate compounds, and higher fatty acids (component (C)) and silicone oil (component (B)) (hereinafter, simply referred to as mixture) at a temperature of 40° C. to 120° C., inorganic oxide powder is added to the silicone oil, and the desired amount is all added.

The silicone oil of the component (B) is a dispersing medium. The component (C) is in such a state of being dissolved or dispersed in the component (B).

The mixture of the component (B) and the component (C) is heated to a predetermined temperature. The heating temperature is 40 to 120° C. When the heating temperature is less than 40° C., the pigment dispersibility is significantly decreased. However, when volatile silicone oil is used, the heating is preferably performed at 60° C. or less in order to prevent the silicone oil from volatilizing. The heating temperature of more than 120° C. is not preferable because the color of yellow iron oxide or black iron oxide changes in the inorganic oxide powder.

The temperature of the mixture is preferably maintained within the above temperature range during addition of the inorganic oxide powder. Preferably, the mixture is heated using a heating unit appropriately so that the temperature is maintained within the above temperature range.

The inorganic oxide powder is preferably gradually added to the mixture. The time for adding the inorganic oxide powder to the mixture is, for example, 1 minute to 100 minutes.

Examples of the dispersion method for obtaining an oil dispersion in the step (1) include a method in which the inorganic oxide powder is added while the silicone oil phase (mixture) is stirred using a homomixer, a disper, or the like. The number of revolutions during stirring is not particularly limited as long as the dispersion proceeds well, but is, for example, 1000 rpm to 5000 rpm.

In terms of the effect of the present invention, preferably, a dispersing agent is not used in the step (1). Examples of the dispersing agent include polyhydroxystearic acid, glyceryl hydroxystearate, polyglyceryl-2 diisostearate, polyglyceryl-2 triisostearate, polyglyceryl-2 tetraisostearate, erythrityl triethylhexanoate, and sorbitan sesquiisostearate.

### Step (2);

The oil dispersed product obtained in the step (1) is further mixed at a temperature of 40° C. to 120° C. to obtain an oil dispersion. The dispersion time in the step (2) is preferably 2 hours or more from the viewpoint of dispersibility of the inorganic oxide powder in the silicone oil, and more preferably 2 hours to 10 hours, and even more preferably 2 hours to 5 hours, from the viewpoint of the saturation of the effect. In the step (2), dispersion may be continued using a homomixer, a disper, or the like. However, for example, a kneading mixer of kneader kneading, Henschel kneading, roll kneading, extruder kneading, or the like, or a wet mixing disperser such as a propeller mixer, a high speed mixer, a dissolver, Altimizer, a wet jet mill, a colloid mill, a mass colloider, a bead mill, a sand mill, a basket mill, or an AD mill, which can apply higher shearing force, is preferably used.

In terms of the effect of the present invention, it is preferable not to use a dispersing agent even in the step (2).

Regarding the temperature conditions at the time of production in the steps (1) and (2), heating is preferably performed so that the temperatures of the mixture and the dispersion reach 40° C. to 120° C. Less than 40° C. is not preferable because the inorganic oxide powder cannot be uniformly dispersed in the silicone oil. Note that, when the volatile silicone oil is used, heating is preferably performed at 60° C. or less in order to prevent the silicone oil from volatilizing. When the temperature is more than 120° C., the color of yellow iron oxide or black iron oxide changes in the inorganic oxide powder, which is not preferable. The heating temperature may be 40° C. to 100° C.

After the steps (1) and (2), additionally performing cooling and, moreover, a dispersion step is preferable because the inorganic oxide powder is further formed into fine particles, and is in a state of being more uniformly dispersed in the oil. Cooling is preferably performed to 40° C. or less, and is preferably performed to room temperature (20° C. to 30° C.). Examples of the cooling include: a method in which the mixture is left to stand while the stirring state is continued; and a method in which a cooling unit is used to cool an oil dispersion while the stirring state is continued.

A dispersing machine used for dispersing after cooling is preferably wet mixing dispersers from the viewpoint of improving the dispersibility of the inorganic oxide powder in the oil dispersion. Among these, a wet bead mill is preferable. Specific examples of the wet mixing disperser are as described above. The dispersing time in the dispersing after cooling is, for example, 15 minutes to 5 hours, and may be 30 minutes to 3 hours.

### (Cosmetic)

Next, the cosmetic according to the present invention will be described. Since the cosmetic of the present invention is a cosmetic having a good feeling because the oil dispersion prepared by the above production method is blended and thus the silicone oil having a good feeling in use is used. In addition, cosmetics such as sunscreens, which have a high solid content concentration but good stability after time has passed, and further have high SPF value and transparency, can be prepared. As specific examples of the cosmetics, they can be used for makeup cosmetics such as foundations, eye shadows, and blushes, or basic cosmetics such as sunscreen cosmetics, hair cosmetics, milky lotions, and creams. The amount of the oil dispersion blended is not particularly limited, and is preferably 0.1 to 90% by mass in the cosmetic.

Furthermore, in the cosmetic of the present invention, components that are normally used in cosmetics, for example, powders, surfactants, oils, gelling agents, polymers, beauty components, moisturizing agents, dyes, preservatives, fragrances, and the like can be used, within the range that the effect of the present invention is not impaired.

As the form of the cosmetic of the present invention, any form, such as the powder form, the milky lotion form, the cream form, the stick form, the solid form, the spray form, or the multi-layer separation type may be used.

### EXAMPLES

Next, the present invention will be described in more detail based on Examples and Comparative Examples of the oil dispersion produced by the production method of the present invention and the cosmetic containing this oil dispersion, but the present invention is not limited to the following Examples.

### Example 1

1.2 g of alkyl-modified and polyglycerin-modified silicone (lauryl polyglyceryl-3 polydimethylsiloxyethyl dimethicone, KF-6105, available from Shin-Etsu Chemical Co., Ltd.) and 38.8 g of dimethicone (KF-96A-6CS, 6 mPa·s, available from Shin-Etsu Chemical Co., Ltd.) were mixed and were heated to 90° C. After it was confirmed that the temperature reached 90° C., 60 g of titanium oxide (MP-1133, available from TAYCA CORPORATION) was added thereto little by little over 10 minutes under stirring with a disper at 2000 rpm. The total amount of titanium oxide was added thereto, followed by further stirring with a disper at 2000 rpm at a temperature of 90° C. for 3 hours, to obtain an oil dispersion of titanium oxide (solid content concentration of titanium oxide 60% by mass).

### Example 2

An oil dispersion of red iron oxide (solid content concentration of red iron oxide 60% by mass) was obtained in the same manner as in Example 1 except that red iron oxide (R-516HP, available from Titan Kogyo, Ltd.) instead of titanium oxide was used.

### Example 3

An oil dispersion of yellow iron oxide (solid content concentration of yellow iron oxide 60% by mass) was obtained in the same manner as in Example 1 except that yellow iron oxide (LL-100HP, available from Titan Kogyo, Ltd.) instead of titanium oxide was used.

### Example 4

An oil dispersion of black iron oxide (solid content concentration of black iron oxide 60% by mass) was obtained in the same manner as in Example 1 except that black iron oxide (BL-100HP, available from Titan Kogyo, Ltd.) instead of titanium oxide was used.

### Example 5

The oil dispersion obtained in Example 1 was left to stand and was cooled to room temperature (25° C.) while stirring was maintained. Then, a wet bead mill (DYNO-MILL, available from SHINMARU ENTERPRISES CORPORATION) was used to disperse and mix it for an hour under the conditions: a circumferential speed of 10 m/s, zirconia beads with ¢ 0.5 mm, and a filling rate of 70%, to obtain an oil dispersion of titanium oxide (solid content concentration of titanium oxide 60% by mass).

### Example 6

4 g of isostearic acid (RADIACID0909, available from Oleon) and 46 g of dimethicone (KF-96L-2CS, 2 mPa·s, available from Shin-Etsu Chemical Co., Ltd.) were mixed and were heated to 60° C. After it was confirmed that the temperature reached 60° C., 50 g of fine particles of titanium oxide (MT-100SA, available from TAYCA CORPORATION) were added thereto little by little over 9 minutes under stirring with a disper at 2000 rpm. After the total amount of the fine particles of titanium oxide was added, a wet bead mill (DYNO-MILL, available from SHINMARU ENTERPRISES CORPORATION) was used to disperse it for 2 hours under the conditions: a circumferential speed of 10 m/s, zirconia beads with ¢ 0.5 mm, a filling rate of 70%, and a temperature of 60° C., to obtain the oil dispersion of the fine particles of titanium oxide (solid content concentration of the fine particles of titanium oxide 50% by mass).

### Example 7

1.2 g of pyrrolidone carboxylic acid-modified silicone (Gransil PCA-C, available from Grant Industries, Inc.) and 38.8 g of dimethicone (KF-96L-2CS, 2 mPa·s, available from Shin-Etsu Chemical Co., Ltd.) were mixed and were heated to 60° C. After it was confirmed that the temperature reached 60° C., 60 g of titanium oxide (MP-1133, available from TAYCA CORPORATION) was added thereto little by little over 10 minutes under stirring with a disper at 2000 rpm. The total amount of titanium oxide was added thereto, followed by further stirring at a temperature of 60° C. for 3 hours, to obtain an oil dispersion of titanium oxide (solid content concentration of titanium oxide 60% by mass).

### Example 8

An oil dispersion of titanium oxide (solid content concentration of titanium oxide 60% by mass) was obtained in the same manner as in Example 7 except that carboxylic acid-modified silicone (ES-5800, available from Dow Toray Co., Ltd.) instead of pyrrolidone carboxylic acid-modified silicone was used.

### Example 9

An oil dispersion of titanium oxide (solid content concentration of titanium oxide 60% by mass) was obtained in the same manner as in Example 7 except that amino-modified silicone (SF8452C, available from Dow Toray Co., Ltd.) instead of pyrrolidone carboxylic acid-modified silicone was used.

### Example 10

2 g of octyltriethoxysilane (triethoxycaprylylsilane, AES-3083, available from Shin-Etsu Chemical Co., Ltd.) and 58 g of decamethylcyclopentasiloxane (KF-995, 4 mPa·s, available from Shin-Etsu Chemical Co., Ltd.) were mixed and were heated to 50° C. After it was confirmed that the temperature reached 50° C., 40 g of fine particles of zinc oxide (MZ-500, available from TAYCA CORPORATION) were added thereto little by little over 8 minutes under stirring with a disper at 2000 rpm. The total amount of the fine particles of zinc oxide was added thereto, followed by further stirring at a temperature of 50° C. for 3 hours, to obtain an oil dispersion of the fine particles of zinc oxide (solid content concentration of the fine particles of zinc oxide 40% by mass).

### Example 11

1.4 g of octyltriethoxysilane (triethoxycaprylylsilane, AES-3083, available from Shin-Etsu Chemical Co., Ltd.) and 28.6 g of decamethylcyclopentasiloxane (KF-995, 4 mPa·s, available from Shin-Etsu Chemical Co., Ltd.) were mixed and were heated to 40° C. After it was confirmed that the temperature reached 40° C., 70 g of zinc oxide (ZnO-S05, available from Sumitomo Osaka Cement Co., Ltd.) was added thereto little by little over 11 minutes under stirring with a disper at 2000 rpm. The total amount of zinc oxide was added thereto, followed by further stirring at a temperature of 40° C. for 3 hours, to obtain an oil dispersion of zinc oxide (solid content concentration of zinc oxide 70% by mass).

### Example 12

1.2 g of isopropyl triisostearoyl titanate (PLENACT KR-TTS, available from Ajinomoto Fine-Techno Co., Inc.) and 38.8 g of diphenylsiloxy phenyl trimethicone (KF-56A, 15 mPa·s, available from Shin-Etsu Chemical Co., Ltd.) were mixed and were heated to 90° C. After it was confirmed that the temperature reached 90° C., 60 g of titanium oxide (MP-1133, available from TAYCA CORPORATION) was added thereto little by little over 10 minutes under stirring with a disper at 2000 rpm. The total amount of titanium oxide was added thereto, followed by further stirring at a temperature of 90° C. for 3 hours, to obtain an oil dispersion of titanium oxide (solid content concentration of titanium oxide 60% by mass).

### Example 13

An oil dispersion of titanium oxide (solid content concentration of titanium oxide 60% by mass) was obtained in the same manner as in Example 12 except that methyl hydrogen polysiloxane (KF-9901, available from Shin-Etsu Chemical Co., Ltd.) instead of isopropyl triisostearoyl titanate was used.

### Example 14

1.5 g of triethoxysilylethyl polydimethylsiloxyethyl dimethicone (KF-9908, available from Shin-Etsu Chemical Co., Ltd.) and 68.5 g of dimethicone (KF-96A-6CS, 6 mPa·s, available from Shin-Etsu Chemical Co., Ltd.) were mixed and were heated to 70° C. After it was confirmed that the temperature reached 70° C., 30 g of fine particles of red iron oxide (TRR-100HP, available from Titan Kogyo, Ltd.) was added thereto little by little over 5 minutes under stirring with a disper at 2000 rpm. The total amount of the fine particles of red iron oxide was added thereto, followed by further stirring at a temperature of 70° C. for 3 hours, to obtain an oil dispersion of the fine particles of red iron oxide (solid content concentration of the fine particles of red iron oxide 30% by mass).

### Example 15

3 g of isostearic acid (RADIACID0909, available from Oleon), 2 g of polyglycerin-modified silicone (polyglyceryl-3 polydimethylsiloxyethyl dimethicone, KF-6106, available from Shin-Etsu Chemical Co., Ltd.), and 45 g of dimethicone (KF-96L-2CS, 2 mPa·s, available from Shin-Etsu Chemical Co., Ltd.) were mixed and were heated to 60° C. After it was confirmed that the temperature reached 60° C., 50 g of fine particles of titanium oxide (MT-100SA, available from TAYCA CORPORATION) was added thereto little by little over 9 minutes under stirring with a disper at 2000 rpm. The total amount of the fine particles of titanium oxide was added thereto, followed by further stirring at a temperature of 60° C. for 3 hours, to obtain an oil dispersion of the fine particles of titanium oxide (solid content concentration of the fine particles of titanium oxide 50% by mass).

### Example 16

1.2 g of palmitic acid (NAA-160, available from NOF CORPORATION) and 38.8 g of dimethicone (KF-96A-6CS, 6 mPa·s, available from Shin-Etsu Chemical Co., Ltd.) were mixed and were heated to 100° C. After it was confirmed that the temperature reached 100° C., 60 g of titanium oxide (MP-1133, available from TAYCA CORPORATION) was added thereto little by little over 10 minutes under stirring with a disper at 2000 rpm. The total amount of titanium oxide was added thereto, followed by further stirring at a temperature of 100° C. for 2 hours, to obtain an oil dispersion of titanium oxide having a solid content concentration of 60% by mass (solid content concentration of titanium oxide 60% by mass).

### Comparative Example 1

An oil dispersion of titanium oxide having a solid content concentration of 60% by mass was obtained in the same manner as in Example 1 except that the temperature was changed from 90° C. to 30° C.

### Comparative Example 2

The treatment agent was not used and 40 g of dimethicone (KF-96A-6CS, 6 mPa·s, available from Shin-Etsu Chemical Co., Ltd.) was heated to 90° C. After it was confirmed that the temperature reached 90° C., 60 g of titanium oxide (MP-1133, available from TAYCA CORPORATION) was added thereto little by little under stirring with a disper at 2000 rpm. The total amount of titanium oxide was added thereto, followed by further stirring at a temperature of 90° C. for 3 hours, to obtain an oil dispersion of titanium oxide having a solid content concentration of 60% by mass.

### Comparative Example 3

The treatment agent was not used and 30 g of decamethylcyclopentasiloxane (KF-995, 4 mPa·s, available from Shin-Etsu Chemical Co., Ltd.) was heated to 40° C. After it was confirmed that the temperature reached 40° C., 70 g of titanium oxide that was surface-treated with octyltriethoxysilane (OTS-2 CR-50, available from DAITO KASEI KOGYO CO., LTD.) was added thereto little by little under stirring with a disper at 2000 rpm. The total amount of the surface-treated titanium oxide was added thereto, followed by further stirring at a temperature of 40° C. for 3 hours, to obtain an oil dispersion of titanium oxide having a solid content concentration of 70% by mass.

### Comparative Example 4

An oil dispersion of titanium oxide having a solid content concentration of 60% by mass was obtained in the same manner as in Example 1 except that squalane instead of dimethicone was used.

Next, the method for evaluating the dispersion stability used in Examples will be described.

The redispersibility (when particles precipitated, it was shaken well) of the sample one day after preparation and the sample stored at 40° C. for one month was visually observed.

Evaluation Criteria after One Day
∘: Dispersed particles were uniform and no aggregation was observed.
Δ: Dispersed particles were almost uniform, but slight aggregation was observed.
x: Dispersed particles were not uniform, and significant aggregation was observed.
   Evaluation Criteria after One-Month Storage
∘: Dispersed particles were uniform and no aggregation was observed.
Δ: Dispersed particles were almost uniform, but slight aggregation was observed.
x: Particles were not redispersed, and precipitation or significant aggregation was observed.

The results obtained by evaluating Examples 1 to 16 and Comparative Examples 1 to 4 by the above evaluation method are described in Table 1.

TABLE 1

| | Example 1 | Example 2 | Example 3 | Example 4 | Example 5 | Example 6 |
|---|---|---|---|---|---|---|
| Dispersibility (after one day) | ∘ | ∘ | ∘ | ∘ | ∘ | ∘ |
| Dispersibility | Δ | Δ | Δ | Δ | ∘ | ∘ |
| (40° C., after one month storage) | | | | | | |

| | Example 7 | Example 8 | Example 9 | Example 10 | Example 11 | Example 12 |
|---|---|---|---|---|---|---|
| Dispersibility (after one day) | ∘ | ∘ | ∘ | ∘ | ∘ | ∘ |
| Dispersibility | Δ | Δ | Δ | Δ | Δ | Δ |
| (40° C., after one month storage) | | | | | | |

| | | | | | Comparative | Comparative |
|---|---|---|---|---|---|---|
| | Example 13 | Example 14 | Example 15 | Example 16 | Example 1 | Example 2 |
| Dispersibility (after one day) | ∘ | ∘ | ∘ | ∘ | x | x |
| Dispersibility | Δ | Δ | ∘ | Δ | x | x |
| (40° C., after one month storage) | | | | | | |

| | Comparative | Comparative |
|---|---|---|
| | Example 3 | Example 4 |
| Dispersibility (after one day) | Δ | x |
| Dispersibility | x | x |
| (40° C., after one month storage) | | |

The products of the present invention, Examples 1 to 16, have good dispersibility one day after production and after one-month storage at 40° C. In addition, it was also found that the products of the present invention have more excellent dispersibility, especially dispersibility after time has passed, than Comparative Examples 1 to 4. It was also found that the oil dispersions of the products of the present invention have more excellent dispersibility after time has passed than the case where the inorganic oxide powder, which has been previously surface-treated, is used (Comparative Example 3).

### Example 17: Oil-Based Foundation

The blending composition shown in Table 2 was dispersed and mixed with a homomixer to obtain an oil-based foundation. The dispersion stability of Example 17 was good.

TABLE 2

| | | Composition |
|---|---|---|
| Component | | (% by mass) |
| (1) | Polyether-modified silicone | 2.0 |
| (2) | Decamethylcyclopentasiloxane | 57.65 |
| (3) | Dimethylpolysiloxane (6cs) | 7.0 |
| (4) | Alkyl acrylate/dimeticone) copolymer | 0.5 |
| (5) | Mica | 5.0 |
| (6) | Tocopherol | 0.05 |
| (7) | Ethylhexyl methoxycinnamate | 5.0 |
| (8) | Nylon powder | 5.0 |
| (9) | Oil dispersion of titanium oxide | 15.0 |
| | of Example 5 | |
| (10) | Oil dispersion of red iron oxide | 0.7 |
| | of Example 2 | |
| (11) | Oil dispersion of yellow iron | 2.0 |
| | oxide of Example 3 | |
| (12) | Oil dispersion of black iron | 0.1 |
| | oxide of Example 4 | |

### Example 18: Sunscreen Cream

A sunscreen cream was obtained using the blending composition shown in Table 3 by the following method. The dispersion stability of Example 18 was good (neither powder aggregation nor precipitation occurred after time has passed).

TABLE 3

| | | Composition |
|---|---|---|
| Component | | (% by mass) |
| (1) | Polyglyceryl diisostearate | 2.0 |
| (2) | Polyether-modified silicone | 4.0 |
| (3) | Oil dispersion of fine particles | 36.0 |
| | of titanium oxide of Example 6 | |
| (4) | Dimethylpolysiloxane (6cs) | 10.0 |
| (5) | Nylon powder | 5.0 |
| (6) | Purified water | 34.5 |
| (7) | 1,3-Butylene glycol | 6.0 |
| (8) | Sodium chloride | 2.0 |
| (9) | Preservative | 0.5 |

### (Production Method)

The components (1) to (5) are mixed together. An aqueous solution of the components (6) to (9) that had been previously mixed and dissolved was added thereto under stirring with a homomixer to obtain the desired water-in-oil sunscreen cream.

Example 17 and Example 18 were cosmetics having good dispersion stability of the pigment (neither powder aggregation nor precipitation occurred after time has passed).

### INDUSTRIAL APPLICABILITY

The oil dispersion produced by the production method of the present invention has a characteristic that inorganic oxide powder that aggregates easily has high dispersibility in silicone oil. In addition, this oil dispersion can provide cosmetics with good dispersion stability, and has great industrial applicability.

The present invention encompasses the following aspects.
1. A production method of an oil dispersion containing the following components
(A) to (C), the production method comprising:
a step (1) of adding a component (A) to a mixture in which a component (C) is dissolved or dispersed in a component (B) while heating the mixture at a temperature of 40° C. to 120° C., and
a step (2) of further mixing a dispersion to which a total amount of the component (A) is added at a temperature of 40° C. to 120° C.;
(A) inorganic oxide powder that is not surface-hydrophobized,
(B) silicone oil, and
(C) at least one compound selected from the group consisting of silicone compounds, organic silicon compounds, organic titanate compounds, and higher fatty acids.
2. The production method of an oil dispersion according to 1.,
wherein a solid content concentration of the component (A) in the oil dispersion is 25% by mass or more.
3. The production method of an oil dispersion according to 1, or 2.,
wherein a kneading mixer or a wet mixing disperser is used for mixing in the step (2).
4. The production method of an oil dispersion according to any one of 1. to 3.,
wherein, after the step (2), the oil dispersion is cooled and is further dispersed using a wet mixing disperser.
5. The production method of an oil dispersion according to 3.,
wherein, after all steps of the production method of 3., the oil dispersion is cooled, and is further dispersed using a wet mixing disperser.
6. The production method of an oil dispersion according to any one of 1. to 5.,
wherein the oil dispersion is an oil dispersion consisting of only the components (A) to (C).
7. A cosmetic comprising:
the oil dispersion produced by the production method according to any one of 1. to 6.

This application is based on Japanese Patent Application No. 2023-091279, filed on Jun. 2, 2023, the disclosure of which is hereby incorporated by reference in its entirety.

1. A production method of preparing an oil dispersion containing the following components (A) to (C), the production method comprising:
a step (1) of adding a component (A) to a mixture in which a component (C) is dissolved or dispersed in a component (B) while heating the mixture at a temperature of 40° C. to 120°, and
a step (2) of further mixing a dispersed product to which a total amount of the component (A) is added at a temperature of 40° C. to 120° C.;
(A) inorganic oxide powder that is not surface-hydrophobized,
(B) silicone oil, and
(C) at least one compound selected from the group consisting of silicone compounds, organic silicon compounds, organic titanate compounds, and higher fatty acids.

2. The production method of an oil dispersion according to claim 1,
wherein a solid content concentration of the component (A) in the oil dispersion is 25% by mass or more.

3. The production method of an oil dispersion according to claim 1,
wherein a kneading mixer or a wet mixing disperser is used for mixing in the step (2).

4. The production method of an oil dispersion according to claim 1,
wherein, after the step (2), the oil dispersion is cooled and is further dispersed using a wet mixing disperser.

5. The production method of an oil dispersion according to claim 3,
wherein, after all steps of the production method according to claim 3, the oil dispersion is cooled, and is further dispersed using a wet mixing disperser.

6. The production method of an oil dispersion according to claim 1,
wherein the oil dispersion is an oil dispersion consisting of only the components (A) to (C).

7. A cosmetic comprising:
the oil dispersion produced by the production method according to claim 1.

8. A cosmetic comprising:
the oil dispersion produced by the production method according to claim 3.

9. A cosmetic comprising:
the oil dispersion produced by the production method according to claim 4.

10. A cosmetic comprising:
the oil dispersion produced by the production method according to claim 5.

11. A cosmetic comprising:
the oil dispersion produced by the production method according to claim 6.
